(19) United States
(12) Patent Application Publication
(10) Pub. No.: US 2011/0105823 A1
(43) Pub. Date: May 5, 2011

(54) Medical Template Device and Method for Use in Positioning Therapeutic Probes at a Target Tissue

(75) Inventors: Gordon W. Single, JR., Queensbury, NY (US); James J. Mitchell, Ballston Spa, NY (US); William C. Hamilton, Queensbury, NY (US)

(73) Assignee: AngioDynamics, Inc.

(21) Appl. No.: 12/609,819

(22) Filed: Oct. 30, 2009

(51) Int. Cl. A61M 36/04; A61B 17/00; A61B 18/14; A61B 18/02; A61M 37/00; A61B 18/18

(52) U.S. Cl. 600/3; 606/1; 606/41; 606/21; 604/506; 606/33

(57) ABSTRACT

Medical template device and method for use in positioning therapeutic probes at a target tissue are provided. The template device includes a frame and a pierceable probe guide secured by the frame. The probe guide has a probe guide pattern such as a grid pattern, wherein the pierceable probe guide is capable of being pierced at any location along its surface by a therapeutic probe. In one embodiment, the pierceable probe guide is a film having a solid surface without any pre-formed apertures. This allows a physician to more accurately position the probes with narrower spacing.

### FIELD OF THE INVENTION

The present invention relates to a medical template device and method for use in positioning therapeutic probes at a target tissue.

### BACKGROUND OF THE INVENTION

Prior art medical template devices, such as those used for brachytherapy and 3D pathologic mapping of the prostate, consist of rows and columns of pre-formed holes typically spaced 5 mm apart. The holes are intended to enable accurate placement of probes, such as electrodes or radioactive seed delivery probes. FIGS. 3A, 4A, and 6A illustrate embodiments of a typical prior art medical template device. These prior art template devices have several problems, which include, but are not limited to, the following.

As shown in FIGS. 3A and 4A, prior art template devices have pre-formed holes 32 through which the probes are inserted. Therefore, it is not possible to space adjacent probes less than the distance (d1) of the spacing between the pre-defined holes, which is typically 5 mm. Furthermore, it is not possible to fine tune the spacing to include positions that are located between the pre-formed holes. The physician is therefore restricted to a pre-determined spacing arrangement. In addition, the maximum diameter of the probe that can be inserted in prior art template devices is limited to the size of the pre-formed holes.

As shown in FIG. 6A, because prior art template devices typically range in thickness from ¼-¾ inch and because the pre-formed holes extend along this same length, the ability of the physician to steer or direct the probe into the desired position during insertion is limited. Probe trajectory is therefore generally limited to a horizontal line relative to the longitudinal axis of the pre-formed holes found in the prior art devices. Also prior art template devices do not allow a physician to control the position of the distal section of the probe once it has been inserted through the pre-formed hole.

In addition, once a probe is removed from a prior art medical template device, it is not possible to determine where it was originally placed without having previously manually noted or marked the location.

Therefore, it is desirable to provide a medical template device and method for use in positioning therapeutic probes at a target tissue which overcomes the problems mentioned above.

### SUMMARY OF THE DISCLOSURE

Throughout the present teachings, any and all of the one, two, or more features and/or components disclosed or suggested herein, explicitly or implicitly, may be practiced and/or implemented in any combinations of two, three, or more thereof, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

Disclosed herein are medical template devices and methods for use in positioning therapeutic probes at a target tissue. In particular, according to one embodiment of the present invention, a medical treatment device includes a frame and a pierceable probe guide secured by the frame and having a probe guide pattern thereon, wherein the pierceable probe guide is capable of being pierced at any location along its surface by a therapeutic probe. In one embodiment, the pierceable probe guide is a film which is devoid of any apertures.

According to one embodiment, the invention includes a method for positioning one or more probes in target tissue using a medical template device. A frame is secured in a fixed position relative to the target tissue, wherein the frame carries a pierceable probe guide secured by the frame and has a probe guide pattern thereon. One or more locations are selected on the pierceable probe guide for inserting the one or more probes. The pierceable probe guide is pierced with the one or more probes at the selected locations. The one or more probes that have pierced the pierceable probe guide are then positioned in the target tissue. The target tissue can be treated by the therapeutic probes by any number of treatment modalities.

### BRIEF DESCRIPTION OF THE DRAWINGS

FIG. 1 is a perspective view of a medical template device assembly of the present invention, which includes a frame, a pierceable probe guide, and mounting pegs.

FIG. 2 is an exploded view of the medical template device of FIG. 1 illustrating the assembly of the individual components.

FIG. 3A is a plan view of a front side of a prior art medical template device with a plurality of pre-defined holes.

FIG. 3B is a plan view of the front side of a medical template device assembly of the current invention.

FIG. 4A is a side view of the prior art grid template of FIG. 3A illustrating a plurality of pre-defined holes shown with two probes positioned through two holes.

FIG. 4B is a side view of the medical template device assembly of FIG. 3B illustrating two probes positioned through the pierceable probe guide.

FIG. 5 is a plan view of the front side of a medical template device assembly of the current invention shown with a plurality of probes positioned through the device.

FIG. 6A is a side view of the prior art medical template device of FIG. 3A illustrating a probe positioned through a hole.

FIG. 6B is a side view of the medical template device assembly of the current invention illustrating a probe positioned through the pierceable probe guide at an angle relative to the surface thereof.

FIG. 6C is a side view of another embodiment of the medical template device assembly of the current invention illustrating a second pierceable probe guide spaced apart from the pierceable probe guide.

FIG. 7 is a side view of the medical template device assembly of the current invention being shown in use during a prostate treatment procedure.

FIG. 8 illustrates a flow chart depicting an embodiment of the method of the present invention.

### DETAILED DESCRIPTION OF THE INVENTION

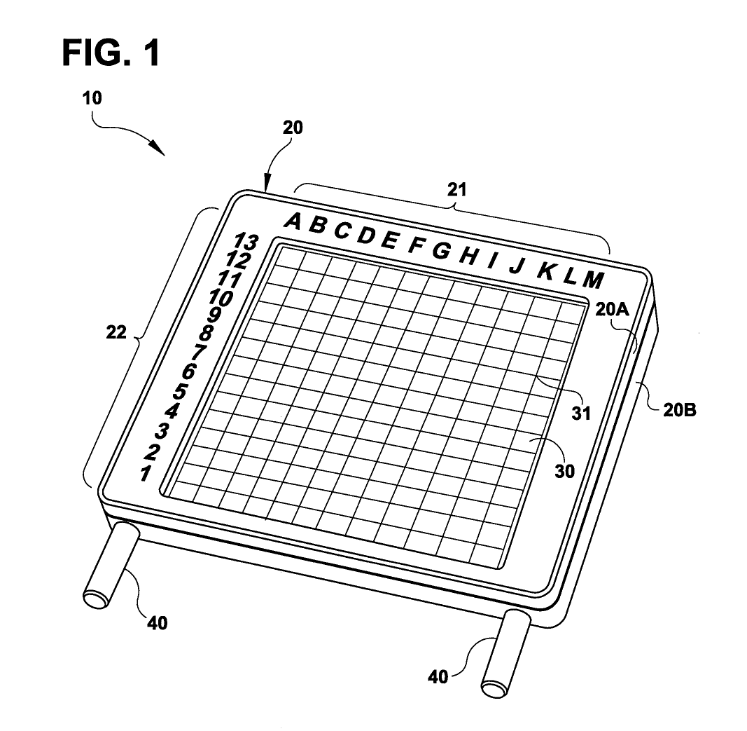

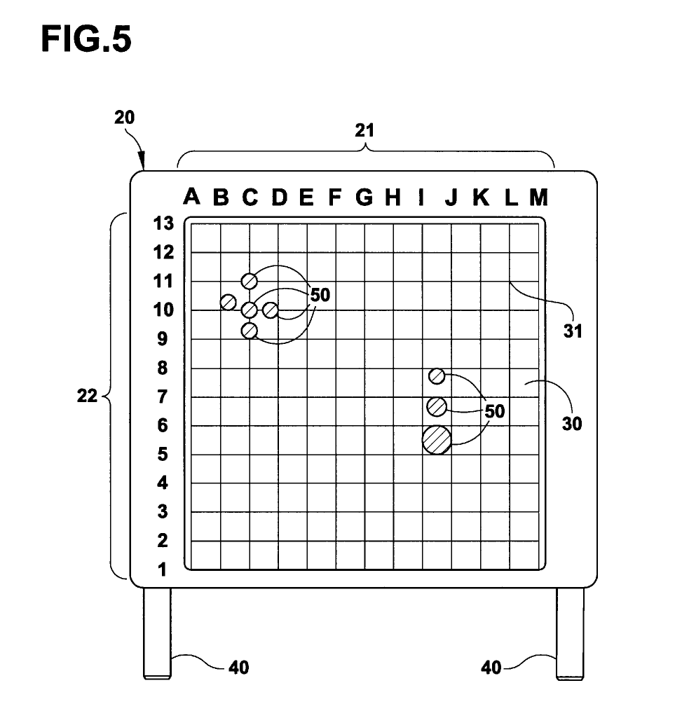

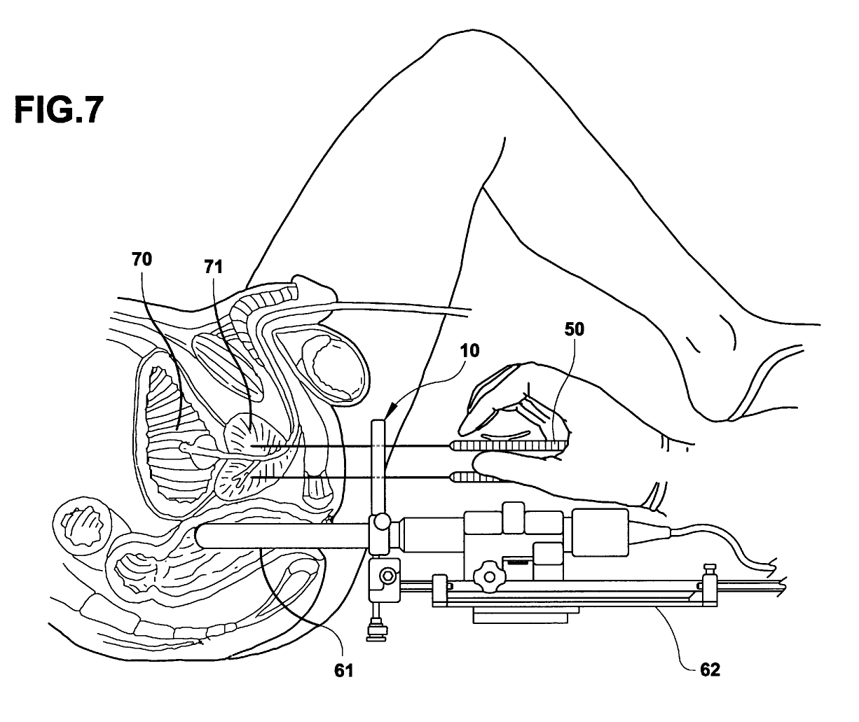

The present invention can be understood by reference to FIGS. 1 through 8. FIG. 1 shows one embodiment of a medical template device for use in positioning therapeutic probes at a target tissue. The device includes a frame 20 which can be comprised of machined plastic or stainless steel, molded plastic, cast steel or any other suitable material. In one embodiment, the frame 20 is about 3 inches by 3.5 inches with an assembled thickness of less than 0.5 inches. A pierceable probe guide 30 is secured by the frame 20 and has a probe guide pattern 31 thereon. The pierceable probe guide pattern can be printed on the surface using biocompatible ink or other markings. The pierceable probe guide 30 is capable of being pierced at any location along its surface by one or more therapeutic probes (see FIG. 5). A pair of mounting pegs 40 is attached to the bottom surface of the frame 20 for attaching the device to an ultrasound stabilizer bracket (see FIG. 7) or other device. Preferably, the probe guide pattern includes a grid pattern. The grid pattern shown includes horizontal and vertical lines which create intersections being spaced 5 mm apart from each other. However, other types of probe guide patterns can be used, including a pattern of dots, cross-hatches, or any other markings which can be used to place probes along the surface of the probe guide 30. In a preferred embodiment, the grid pattern 31 correlates to a grid shown on an ultrasound display monitor (not shown). Preferably, the frame 20 includes indicia 21, 22 for at least some of the x-axis and y-axis lines, respectively, of the grid pattern 31. The indicia can be any combination of letters, numbers, or any other symbols which can be used to designate locations on the pierceable probe guide 30.

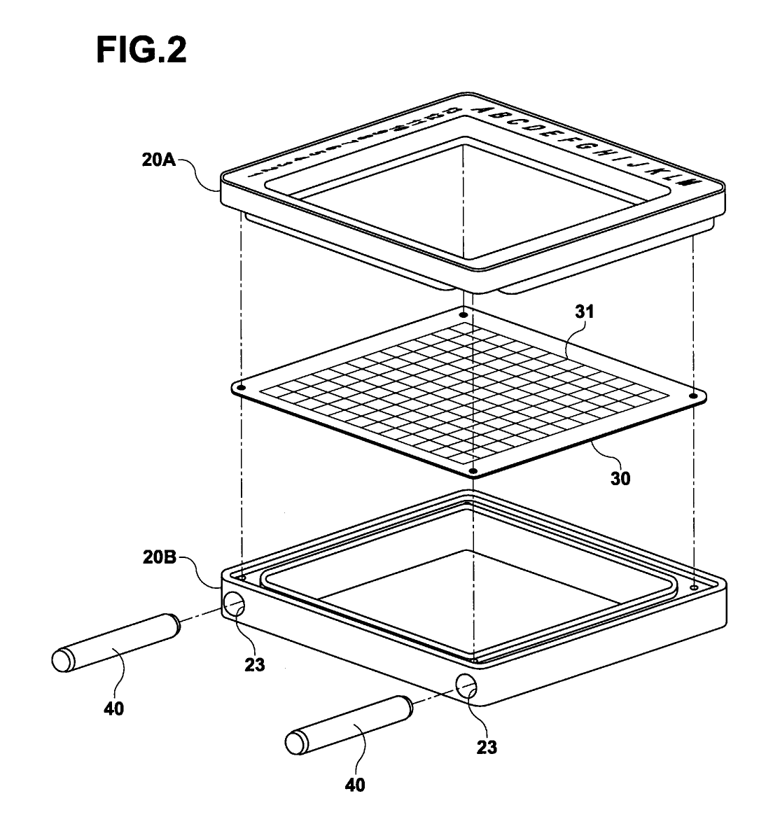

FIG. 2 is an exploded view of the medical template device of FIG. 1 illustrating the assembly of the individual components. According to the embodiment shown, the frame 20 includes a first section 20A and a second section 20B, wherein the pierceable probe guide 30 is secured between the first and second sections. Screws or other attachment mechanisms (not shown) can be used to attach the first and second sections of the frame together. Alternatively, an adhesive, such as epoxy, can be used to bond the sections of the frame together. While the frame 10 is shown as having four sides, other embodiments are possible. For example, an L-shaped frame with two sides may be used to hold the probe guide sheet 30. The frame can have any shape, such as circular.

In one embodiment, the pierceable probe guide includes a film. The film can be a plastic film, which forms a continuous plane of material. In another embodiment, the pierceable probe guide includes a woven mesh of plastic material, having small closely spaced holes which are formed between each of the weaves of the material. Therefore, one advantage of the woven mesh material is that the therapeutic probe would not require a sharp needle tip as there is nothing to pierce. For example, probes having blunt tips can be inserted through the woven mesh material.

The pierceable probe material which is used should limit translational movement and exhibit high tear resistance. Examples of suitable material for the pierceable probe guide include, but are not limited to, TYVEK™, MYLAR™, and NYLON. The minimum thickness of the pierceable probe guide is dependent upon its resistance to tearing. In one embodiment the pierceable probe guide is a film which has a thickness of 0.004 inches or greater. The maximum thickness of the pierceable probe guide is dependent upon its puncture force and its ability for allowing the therapeutic probe to rotate freely about the puncture hole so as to permit the probe to be steered at a desired angle relative to the surface of the pierceable probe guide.

In another embodiment, the pierceable probe guide can include a combination of a woven mesh and a film. For example, the woven mesh can be provided with a probe guide pattern and the film can be used to provide the probe placement history via the holes created therein. Even though this embodiment would include a combination of a woven mesh and a film, the physician is still able to steer the probes to the target tissue.

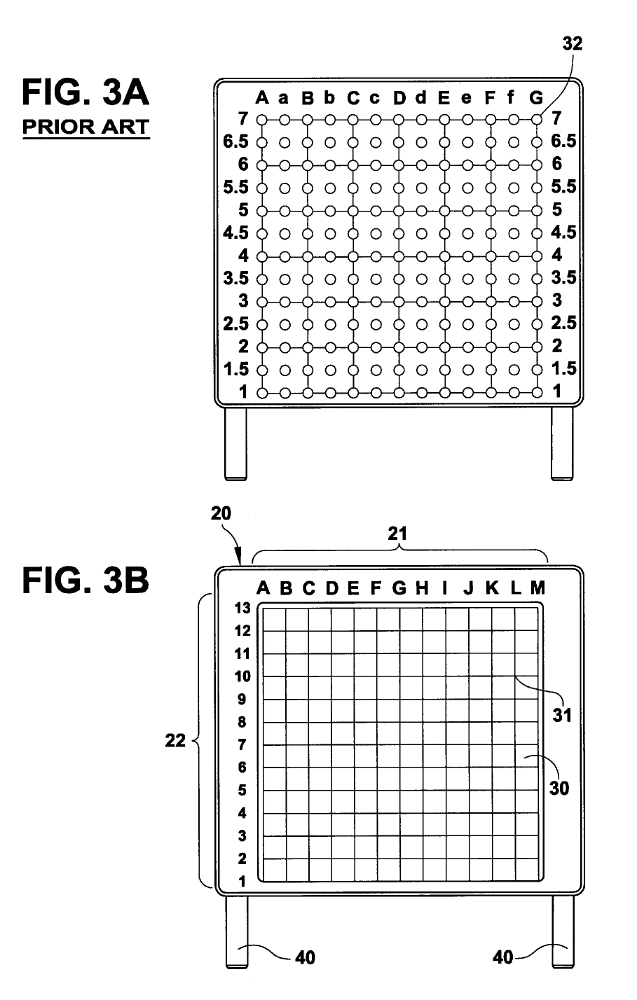

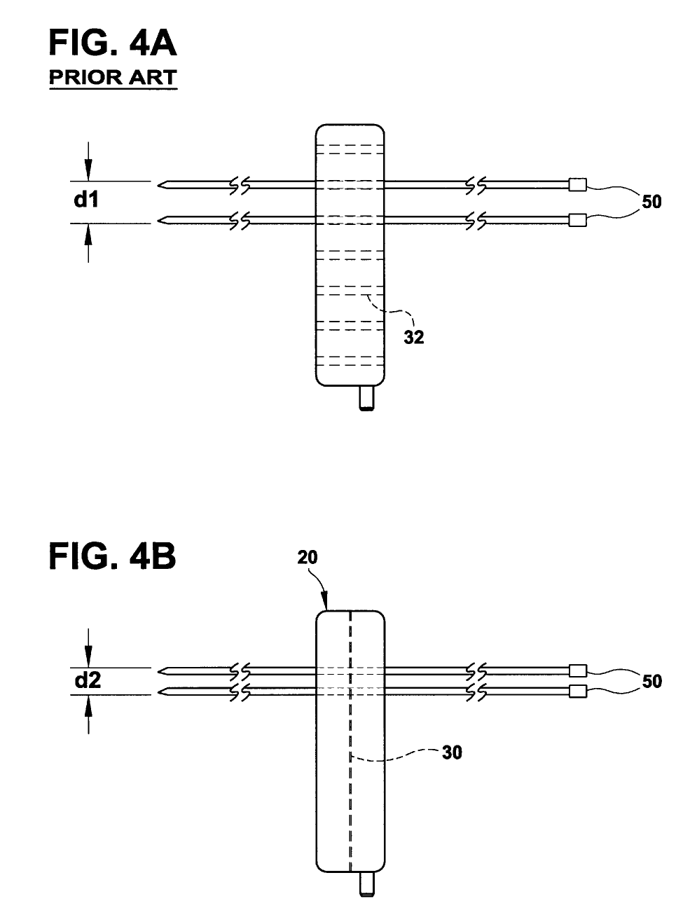

Compared to the prior art medical template devices (see FIG. 3A), the surface of the pierceable probe guide 30 of the present invention is devoid of any pre-formed apertures as shown in FIG. 3B. As discussed above, with prior art devices (see FIG. 4A), the prior art template devices have pre-formed holes 32, and, therefore, it is not possible to achieve a spacing between adjacent probes 50 of less than the pre-defined spacing between the holes (d1), which is typically 5 mm. The physician is therefore restricted to a pre-determined spacing arrangement. On the other hand, as shown in FIG. 4B and in accordance with the present invention, the physician may place therapeutic probes 50 according to the needs of the treatment rather than being restricted to a pre-determined spacing arrangement. For example, if necessary the physician can place the probes 50 closely to one another as shown by the distance (d2) in FIG. 4B.

FIG. 5 is a plan view of the front side of a medical template device assembly of the current invention shown with a plurality of probes positioned through the pierceable probe guide. A cluster of five closely spaced probes 50 is shown near the top left quadrant of the pierceable probe guide in FIG. 5. The locations of the probes 50 were selected by the physician and were placed without any restrictions.

Another advantage of the present invention is that the pierceable probe guide can accommodate a variety of probes 50 having many different diameters. Because the pierceable probe guide is devoid of any apertures and can be pierced at any location along its surface, any diameter probe can be inserted through the pierceable probe guide. Near the lower right quadrant of FIG. 5, a row of three different probes are shown, each having a larger diameter than the next.

As also discussed above, once a probe is removed from a prior art medical template device, it is not possible to determine where it was originally placed without having previously manually noted or marked the location. On the other hand, with the present invention the physician is able to easily identify each of the locations in which the probes were previously placed by simply identifying the presence of each hole through the surface of the pierceable probe guide. If holes are present then they represent the locations where the probes were previously inserted through the pierceable probe guide.

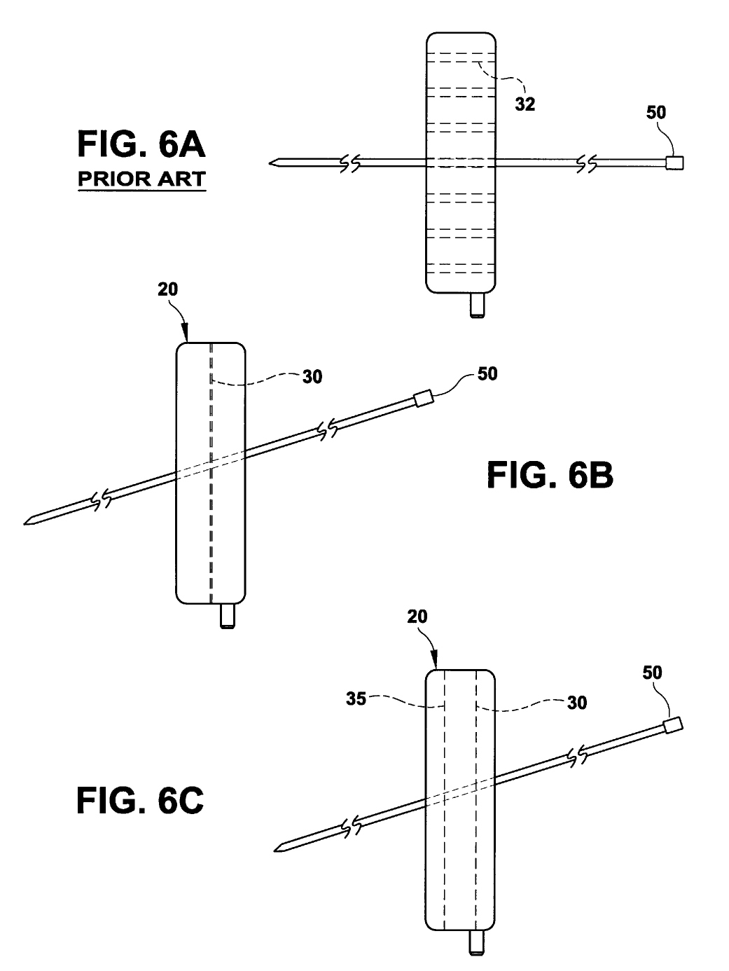

As discussed above, prior art template devices typically limit the ability of the physician to steer or direct the probe 50 into the desired position during insertion and also do not allow the control of the position of the distal end of the probe after it is inserted through the device (see FIG. 6A). Probe trajectory is therefore generally limited to a horizontal line relative to the longitudinal axis of the pre-formed holes found in the prior art devices. By way of comparison, as shown in FIG. 6B, the present medical template device allows the physician the ability to insert the probe 50 at any angle and precisely position the probe 50 in the desired location of the target tissue (not shown).

FIG. 6C is a side view of another embodiment of the medical template device assembly of the current invention illustrating a second pierceable probe guide 35 spaced apart from the pierceable probe guide 30. Preferably, the second pierceable probe guide 35 is made of the same material as the pierceable probe guide 30 and includes a probe guide pattern. The second pierceable probe guide 35 can be positioned substantially parallel to the pierceable probe guide 30. The second pierceable probe guide 35 is capable of being pierced at any location along its surface by a probe 50.

In one embodiment, the probe guide pattern of the pierceable probe guide 30 differs from the probe guide pattern of the second pierceable probe guide 35 and the pierceable probe guide 30 is substantially transparent. In this embodiment, the physician can plan a probe trajectory in advance and use the probe guide patterns of each pierceable probe guide, respectively, to execute the desired trajectory of the probe while it is being inserted into the target tissue. For example, the physician can calculate the angle at which the probe is to be placed and then use the indicia of the second pierceable probe guide 35 relative to the pierceable probe guide 30 to place the probe at the correct angle. In one embodiment, the second pierceable probe guide 35 can include indicators which are positioned relative to the probe guide pattern of the pierceable probe guide 30 in such a way as to represent pre-determined angles of insertion. The angle calculated by the indicators would of course depend on the distance between the pierceable probe guide 30 and the second pierceable probe guide 35.

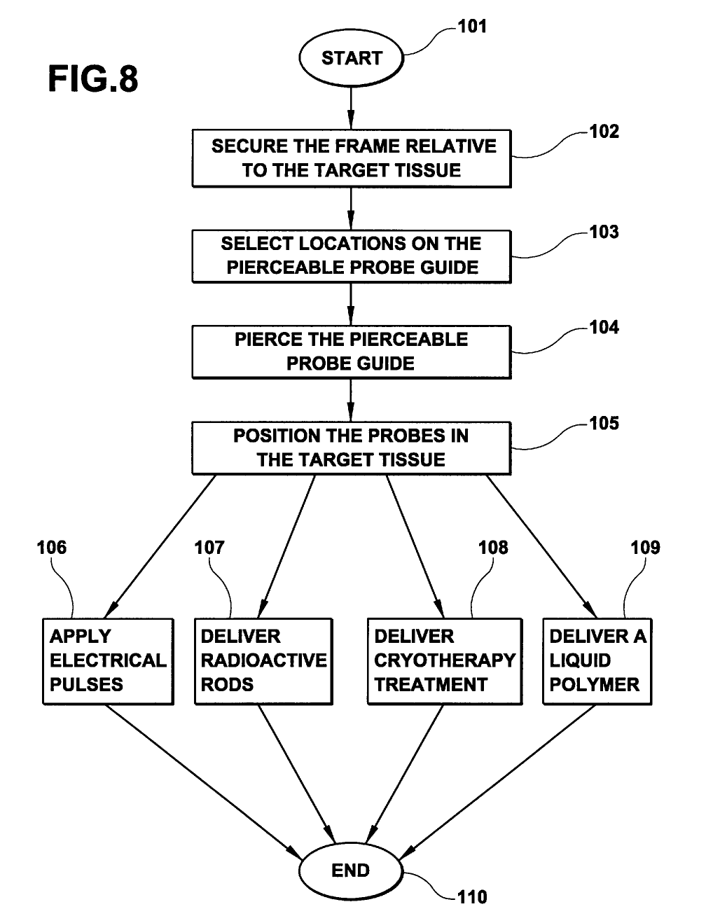

Referring now to FIGS. 7-8, one embodiment of the method for positioning one or more probes in target tissue using a medical template device is disclosed. In the embodiment shown in FIG. 7, the target tissue is prostate tissue 71. The present device can be used for positioning probes in any other type of target tissue including, but not limited to, breast tissue, esophageal tissue, or any other living tissue. The target tissue is detected and its location is determined using ultrasound imaging or the like. The method starts at (101). The frame is secured in a fixed position relative to the target tissue, wherein the frame carries a pierceable probe guide secured by the frame and having a probe guide pattern thereon (102). Referring to FIG. 7, the frame can be secured to an ultrasound stabilizer bracket of an ultrasound stepper 62. The ultrasound stepper is attached to an ultrasound probe 61 that is inserted into the rectum of the patient. The physician selects one or more locations on the pierceable probe guide for inserting the one or more probes (103). The locations are selected depending on the location of the target tissue and the type of procedure being administered. Next, the physician pierces the pierceable probe guide with the one or more probes at the selected locations (104). The physician positions, in the target tissue, the one or more probes that have pierced the pierceable probe guide (105). An imaging device, such as ultrasound, can be used to view the location of the one or more probes as they are being positioned. If the device includes a second pierceable probe guide as explained above, then the physician pierces the second pierceable probe guide prior to the step of positioning the probes.

Once the probes are positioned, the probes can deliver any of a number of therapeutic treatments to the target tissue. In one embodiment, the probes can be electrodes which apply electrical pulses between the probes in an amount sufficient to subject cells within the target tissue to irreversible electroporation (106). The electrical pulses can have an amplitude in the range of 500 Volt/cm and 1500 Volt/cm and can have a duration in a range of 50 microseconds and 150 microseconds. In another embodiment, the probes can be needles which deliver radioactive materials such as radioactive rods into the target tissue for treating cancer via brachytherapy (107). In another embodiment, the probes are cryoneedles and can deliver cryotherapy treatment to the target tissue (108) which freezes the target tissue. In one embodiment, an Argon gas is circulated through the tips of the cryoneedles. The freezing gas creates ice balls on the tips of the needles. The freeze cycle lasts about 10 minutes or until the temperature reaches negative 40 degrees Celsius. Then, the target tissue is thawed. This freeze-thaw cycle can be repeated several times. In another embodiment, the probes can be used to deliver radiofrequency energy to the target tissue.

In another embodiment, the probes can be used to deliver a curable liquid polymer to the target tissue (109). Once the polymer is delivered, it can be cured (e.g., by ultraviolet light) to form a solid substance to fill any void that is created from removing tissue in a target zone. In another embodiment, the probes are used for 3D pathologic mapping of the prostate or other area of the patient.

The probes are then removed from the target tissue and the procedure is complete (110).

The present invention affords several advantages as discussed herein. The probes can be spaced as close or as far apart as desired without having to follow any preselected locations. The device can accept probes of various diameters. The device allows the probes to be inserted at an angle to the surface. The physician can easily determine the locations where previous probes were placed. Also, the physician can adjust the position of the probe after it is inserted through the pierceable probe guide.

The above disclosure is intended to be illustrative and not exhaustive. This description will suggest many modifications, variations, and alternatives which may be made by persons having ordinary skill in this art without departing from the scope of the invention. Those familiar with the art may recognize other equivalents to the specific embodiments described herein. Accordingly, the scope of the invention is not limited to the foregoing specification.

What is claimed is:

1. A medical template device for use in positioning therapeutic probes at a target tissue comprising:
a frame; and
a pierceable probe guide secured by the frame and having a probe guide pattern thereon, wherein the pierceable probe guide is capable of being pierced at any location along its surface by a therapeutic probe.

2. The medical template device of claim 1, wherein:
the pierceable probe guide includes a film; and
the probe guide pattern includes a grid pattern.

3. The medical template device of claim 1, wherein the pierceable probe guide includes a plastic film.

4. The medical template device of claim 1, wherein the pierceable probe guide includes a woven mesh of plastic material.

5. The medical template device of claim 1, wherein a surface of the pierceable probe guide is devoid of any apertures.

6. The medical template device of claim 1, further comprising:
a second pierceable probe guide secured by the frame and having a probe guide pattern thereon, wherein the second pierceable probe guide is capable of being pierced at any location along its surface by the therapeutic probe; and
wherein the second pierceable probe guide is positioned substantially parallel to the pierceable probe guide.

7. The medical template device of claim 7, wherein the probe guide pattern of the pierceable probe guide differs from the probe guide pattern of the second pierceable probe guide.

8. The medical template device of claim 7, wherein the pierceable probe guide is substantially transparent.

9. The medical template device of claim 1, further comprising:
a pair of mounting pegs attached to the frame for attaching the device to an ultrasound stabilizer bracket.

10. The medical template device of claim 1, wherein:
the probe guide pattern includes a grid pattern; and
the frame includes indicia for at least some of the x-axis and y-axis lines of the grid pattern.

11. The medical template device of claim 1, wherein the frame includes first and second sections and wherein the pierceable probe guide is secured between the first and second sections.

12. A medical template device for use in positioning therapeutic probes at a target tissue comprising:
a frame; and
a probe guide film secured by the frame and having a probe guide pattern thereon, wherein the probe guide film is devoid of any apertures and is capable of being pierced at any location along its surface by a therapeutic probe.

13. The medical template device of claim 12, wherein the probe guide film includes a plastic film.

14. The medical template device of claim 12, wherein the probe guide film includes a woven mesh of plastic material.

15. The medical template device of claim 12, further comprising:
a second probe guide film secured by the frame and having a probe guide pattern thereon, wherein the second probe guide film is capable of being pierced at any location along its surface by the therapeutic probe; and
wherein the second probe guide film is positioned substantially parallel to the probe guide film.

16. The medical template device of claim 12, further comprising:
a pair of mounting pegs attached to the frame for attaching the device to an ultrasound stabilizer bracket.

17. The medical template device of claim 12, wherein:
the probe guide pattern includes a grid pattern; and
the frame includes indicia for at least some of the x-axis and y-axis lines of the grid pattern.

18. The medical template device of claim 12, wherein the frame includes first and second sections and wherein the pierceable probe guide is secured between the first and second sections.

19. A method for positioning one or more probes in target tissue using a medical template device comprising:
securing a frame in a fixed position relative to the target tissue, wherein the frame carries a pierceable probe guide secured by the frame;
selecting one or more locations on the pierceable probe guide for inserting the one or more probes;
piercing the pierceable probe guide with the one or more probes at the selected locations; and
positioning, in the target tissue, the one or more probes that have pierced the pierceable probe guide.

20. The method of claim 19, wherein the step of positioning includes:
advancing the one or more probes into the target tissue while using an imaging guide device to view the location of the one or more probes in the target tissue.

21. The method of claim 19, wherein the one or more probes include one or more electrodes and the method further comprises the step of:
applying through the positioned electrodes electrical pulses in an amount sufficient to subject cells within the target tissue to irreversible electroporation.

22. The method of claim 21, wherein the step of applying includes applying electrical pulses whose amplitude is in the range of 500 Volt/cm and 1500 Volt/cm and whose duration is in a range of 50 microseconds and 150 microseconds.

23. The method of claim 19, wherein the one or more probes include one or more needles and the method further comprises the step of:
delivering radioactive material through the positioned needles and into the target tissue for treating cancer via brachytherapy.

24. The method of claim 19, further comprising the step of:
delivering cryotherapy treatment through the one or more probes.

25. The method of claim 19, further comprising the step of:
delivering a curable liquid polymer through the one or more probes and into the target tissue.

26. The method of claim 19, further comprising the step of:
applying radio frequency energy through the positioned probes.

27. The method of claim 19, wherein the step of positioning includes:
inserting the probes at an angle relative to the surface of the pierceable probe guide.

28. The method of claim 19, wherein the frame carries a second pierceable probe guide having a probe guide pattern and wherein the second pierceable probe guide is positioned substantially parallel to the pierceable probe guide, and the method further comprises the step of:
piercing the second pierceable probe guide with the one or more probes prior to the step of positioning.

29. The method of claim 19, wherein:
the pierceable probe guide has a probe guide pattern; and
the step of selecting includes selecting one or more locations on the pierceable probe guide using the probe guide pattern as a guide.
